(19) United States
(12) Patent Application Publication
(10) Pub. No.: US 2014/0330307 A1
(43) Pub. Date: Nov. 6, 2014

(54) Surgical Passer and Retrieval Device

(71) Applicant: Dennis L. Steffen, Tavernier, FL (US)

(72) Inventor: Dennis L. Steffen, Tavernier, FL (US)

(21) Appl. No.: 13/874,755

(22) Filed: May 1, 2013

(51) Int. Cl. A61B 17/28

(52) U.S. Cl. CPC A61B 17/28
USPC 606/205

(57) ABSTRACT

A manual surgical passer and retrieval Device and method are provided. The Device includes a flexible dual shaft passer and retriever. The Device is especially configured to aid in the retrieval and passage of various elements such as sutures encountered during a surgical procedure.

### CROSS REFERENCES TO RELATED APPLICATIONS

This application claims the benefit under 35 U.S.C. §119(e) of U.S. Non-Provisional application Ser. No. 12/904,221, filed on Oct. 14, 2010, the complete disclosure of which is incorporated herein by reference in its entirety.

### FIELD OF THE INVENTION

The present invention described herein is relates to a flexible light weight surgical Device specifically designed and proportioned for the retrieval and passage of various elements encountered during a surgical procedure. The Device is not limited to and may be used to retrieve such elements as; sutures, dips, cables, soft tissues, severed or torn tendons, and torn muscles etc. Although the present specification describes the use of the Device in the retrieval of a lacerated flexor tendon in the hand, additionally the Device may be used in any number of surgical procedures as a retrieval Device i.e. braided fracture fixation cables or a passer Device i.e. suture passer. Furthermore described herein the Device may be proportioned as would be required for other applications.

### INTRODUCTION

Related to the application of the Device in the hand the conventional retrieval Devices are not specifically designed to address the issues and requirements associated with the repair of a lacerated flexor tendon i.e. user friendly, correctly proportioned, correct amount of flex, weight of the Device, and reuse of the Device, surgeon would greatly benefit from a Device with these characteristics. In addition to having technical expertise, experienced hand surgeons must have precise knowledge of flexor tendon anatomy to guide appropriate treatment of injuries to the flexor tendon system. This Device will greatly benefit the surgeon in providing a Device specifically developed to address the fore mentioned requirements. The Device will have a significant effect on reduction of the time needed to complete such procedures.

Regarding surgery of lacerated flexor tendon in the hand the flexor tendon moves within and are incased in a fibrous like material known as the sheath which is attached to the bones in the finger. When the tendon has been severed one end will typically retract like a rubber band while the other end will remain at the laceration site. One commonly used method of joining/securing the free ends of a completely severed flexor tendon back together is by the use of sutures. After the two ends of the lacerated tendon have been identified, the free end of the tendon which is to be brought back to the repair site is pulled up though an opening created by the surgeon; a suture is then connected to this end. The retrieval Device is inserted at the laceration/repair site it is then advanced through the tendon sheath lumen to the site of the previously sutured end. Once the suture is secured to the tendon end then the suture is secured to the distal aperture of the Device and is then pulled back thought the sheath lumen by retracting the Device bringing the suture and then the free end of the sutured tendon back through the sheathing and positioning it adjacent to the other severed free end of the tendon at the repair site. Once at the repair site the Device is released from the suture and removed, thus allowing the surgeon to complete the joining of the two ends and completing the procedure.

### BRIEF DESCRIPTION

The invention provides a flexible light weight Device to aid in tendon repair and other surgical procedures encountered in acute injury. The Device formed from a single continuous flexible light weight element has a dual tapered shaft portion and a permanently joined proximal finger pull region sized to except a fingertip i.e. forefinger. The tapered shafts have a convex upper surface and an opposed convex under surface with a U shaped distal tip. The finger pull on the proximal end of the shaft provides for a slip free method of gripping the Device while in use. Different from current devices used in other area of the anatomy which do not provide the proper combination of stiffness and flexibility or the correct shape and proportion to match the anatomy in my invention the dual shafts have a permanently formed longitudinal arc extending from the extreme apex of the finger pull region on the proximal end to the extreme apex of the tip region on the distal end. The arc which is well suited to rest in the sheath lumen of a finger in a relaxed state during the procedure, the arc also allows for easier insertion into the sheath lumen while providing a more ergonomic and self-orienting shape better suited to the anatomy of a finger in a relaxed prone position. The arc is also ergonomically acceptable for use of the Device in other parts of the anatomy such as areas around the bones of the upper and lower extremities. The U shape in the distal region is sized to easily accommodate a suture or other attachment Devices commonly used in surgical procedures. The length and thickness of the shafts are also sized proportionately to provide the right combination of stiffness and flexibility, and can be sized for smaller areas such as the hand and larger for areas such as the upper and lower extremities thus allowing for minimal trauma during the surgical procedure.

This Device described in detail is proposed for the a traumatic retrieval of sutures within the flexor tendon sheath. The Device is intended but not limited to specifically, to address the anatomy of the hand and fingers and the requirements of the flexor tendons within the confines of the flexor tendon sheath during surgical repair of lacerated tendons.

Anatomy in the distal palm and the entire finger, the flexor tendon slides within a fibro-osseous lumen. The fibrous component of the lumen is referred to as the flexor tendon sheath. The sheath is made of individual lumen called pulleys which coincide with the finger's joints. The repair of a lacerated tendon within the flexor tendon sheath is a technical challenge to any surgeon. It is frequently advantageous to suture the transected end of a tendon and then retrieve the suture first. Once the suture is retrieved, the tendon can be pulled/feed through the sheath to the site of the repair. Most surgeons utilize less than adequate Devices such as rubber or plastic catheters of varying sizes i.e. 5 French for tendon or suture retrieval as portrayed in the following publications (Sourmelis S G. McGrouther D A: Retrieval of the retracted flexor tendon. J Hand Surg 1987; 12B:109-111). Some surgeons recommend a loop made of malleable surgical wire; the wire loop is twisted many times to give it the shape to be used as a suture retriever. (B. Jagannath Kamath, Praveen Bhardwaj, J Hand Surg 2007; 32A:269-273). This proposed Device has numerous advantages over the current Devices and fabricated substitutions as described above and or proposed in other inventions. This Device is capable of being specifically sized for the application in the hand additionally it is a self-introducing Device with the correct combination of curvature, stiffness and flexibility which does not require any additional assistance such as threading by use of a forceps as do some of the earlier described devices/substitutions or similar devices made from polymeric materials. The Device also has the advantages of being simple to clean and capable of re-sterilization which makes it reusable which results in a more cost effective Device then other disposable options.

In one embodiment where the Device is proportioned for use in the hand, the invention provides a Device which has a proportionately sized and permanently joined closed radius end points at the expanded proximal end which provides a finger pull that is non-intrusive and allows the surgeon to advance or retract the Device either by gripping the finger pull between the thumb and forefinger or by slipping the tip of the forefinger through the open portion of the finger pull. The region between the proximal finger pull and the distal tip has a tubular shaped tapered dual shaft portion with a convex upper surface and convex under surface. The shaft tapers in thickness from the center point of the distal tip to the start of the proximal finger pull region, thinner at the tip to thicker at the finger pull. The shaft also has a U shape tip in the most distal region. The dual shafts have a permanent arc extending from the most distal edge of the U shape tip to the most proximal edge of the finger pull. The advantages of the permanently formed arced shafts is it enables the Device to be self-orienting and fit and rest naturally in the anatomy of the finger when in a relaxed prone position and also to provide an ergonomic shape for a more natural insertion approach as the Device is introduced and advanced into the flexor tendon sheath lumen where the sheath is empty as in the area of a lacerated and retracted tendon. In addition to having a U shape tip to aid in finding its way as it is introduced and advanced into the flexor tendon sheath lumen and where the sheath surrounds a tendon its design allows the Device to fit very naturally without trauma to the tendon or the sheath surrounding the tendon as it is introduced and advanced into the flexor tendon sheath lumen, and where the flexor digitorum superficialis hiatus is intact the U shape tip is designed to allow for easy entry into the hiatus of the flexor digitorum superficialis without trauma to this structure or the mesotenon (the connective tissue sheath attaching a tendon to its fibrous sheath). This is relevant when repairing the flexor digitorum profundus tendon.

With respect to the dual tubular shaped shaft configuration which provides a naturally occurring curvature element in its longitudinal axis to match the convex outline of the volar aspect of the proximal and middle phalanx where the tightest pulleys exists which makes retrieval of a severed tendon more challenging.

Other advantages and a better appreciation of the specific adaptations, variations, and physical attributes of the invention will be gained upon an examination of the following detailed description of the invention, taken in conjunction with the accompanying drawings.

### BRIEF DESCRIPTION OF THE DRAWINGS

The invention may be better understood and appreciated by reference to the detailed description of specific embodiments presented herein in conjunction with the accompanying drawings of which:

FIG. 1 is a top plan view of the Device configuration with the U shape tip and the permanently joined radius end points of the finger pull;

FIG. 2 is a side plan view of the Device configuration with the U shape tip and the permanently joined radius end points of the finger pull and with the arced shafts;

FIG. 3 depicts a cross sectional view of the Device's dual shaft configuration this illustrates the naturally occurring convex upper surface and convex under surface;

FIG. 4 illustrates a perspective view of the suture passer configuration with the U shape tip and permanently joined radius end points of the finger pull;

FIG. 5 illustrates a partial perspective view of the Device configuration with a filled finger pull suitable for thumb and index finger gripping;

FIG. 6 illustrates a partial perspective view of the Device configuration with a partial tubular sleeve over joined ends of the finger pull suitable for thumb and index finger gripping;

FIG. 7 illustrates a partial perspective view of the Device configuration with welded joined ends of the finger pull suitable for thumb and index finger gripping;

FIG. 8 illustrates a partial perspective view of the Device configuration with a full tubular sleeve over joined ends of the finger pull suitable for thumb and index finger gripping;

FIG. 9 illustrates a partial perspective view of the Device configuration with a full metal cover over joined ends of the finger pull suitable for thumb and index finger gripping;

FIG. 10 illustrates a partial side plan view of the Device as applied in retrieval of a sutured attached to the free end of a lacerated flexor tendon;

FIG. 11 illustrates a partial top plan view of the Device as applied in retrieval of a sutured attached to the free end of a lacerated flexor tendon;

### DETAILED DESCRIPTION

A passer/retriever Device embodying the principles of the invention is provided. The Device includes a passer/retriever.

Before any embodiments of the invention are explained in detail, it is to be understood that the invention is not limited in its application to the details of the structure retrieval and function set forth in the following description or illustrated in the appended drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. “Comprising” also encompasses the terms “consisting of” and “consisting essentially of.” The use of “consisting essentially of” means, e.g., that a method may include additional steps, but only if the additional steps do not materially alter the basic and novel characteristics of the claimed method. Unless specified or limited otherwise, the terms “joined”, “connected”, and “coupled” and variations thereof are used broadly and encompass both direct and indirect joined, connections, and couplings. Further, “joined”, “connected” and “coupled” are not restricted to physical or mechanical connections or couplings.

No admission is made that any reference, including any patent or patent document, cited in this specification constitutes prior art. In particular, it will be understood that unless otherwise stated, reference to any document herein does not constitute an admission that any of these documents forms part of the common general knowledge in the art in the United States or in any other country. Any discussion of the references states what the author asserts and the applicant reserves the right to challenge the accuracy and pertinence of any of the documents cited herein.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

Unless otherwise noted, technical terms are used according to conventional usage. However, as used herein, the following definitions may be useful in aiding the skilled practitioner in understanding the invention. Such definitions shall be applied, unless a different definition is given in the claims or elsewhere in this specification.

As used herein, the term “aperture” is meant to refer to an aperture of a passer/retriever Device that is configured to accommodate sutures and other surgical attachment Devices of varying sizes.

As used herein, the term “finger pull” is meant to refer to permanently closed or joined end points of an aperture of a passer/retriever Device that is configured to accommodate the tip portion of the index finger of varying sizes.

As used herein, the term “over-mold” is meant to refer to a material permanently joined to the Device by means of an injection molding process.

In view of the foregoing disadvantages inherent in conventional suture passers/retrievers, the invention provides a novel Device for repair of flexor tendons. The invention provides an instrument specifically designed and proportioned for use in this area. However the Device is capable of being proportioned for use in other areas of the anatomy such as upper and lower extremities e.g. the distal bicep tendon repair.

The Device proposed here is unique to the anatomy of the hand but could easily be adapted for other areas of the anatomy.

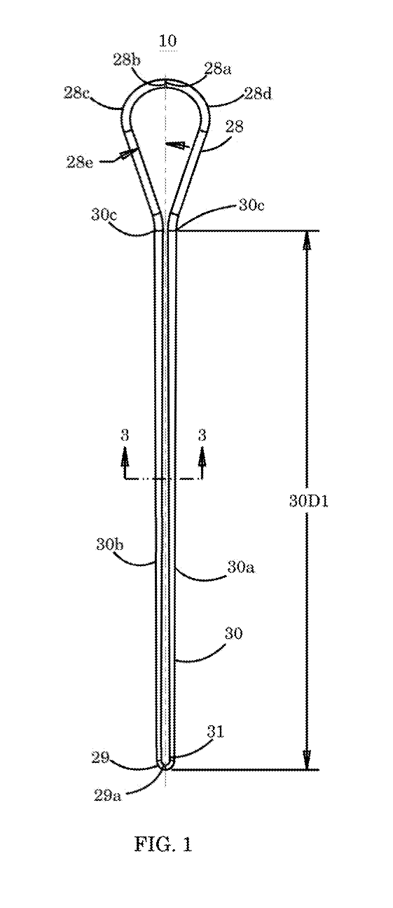

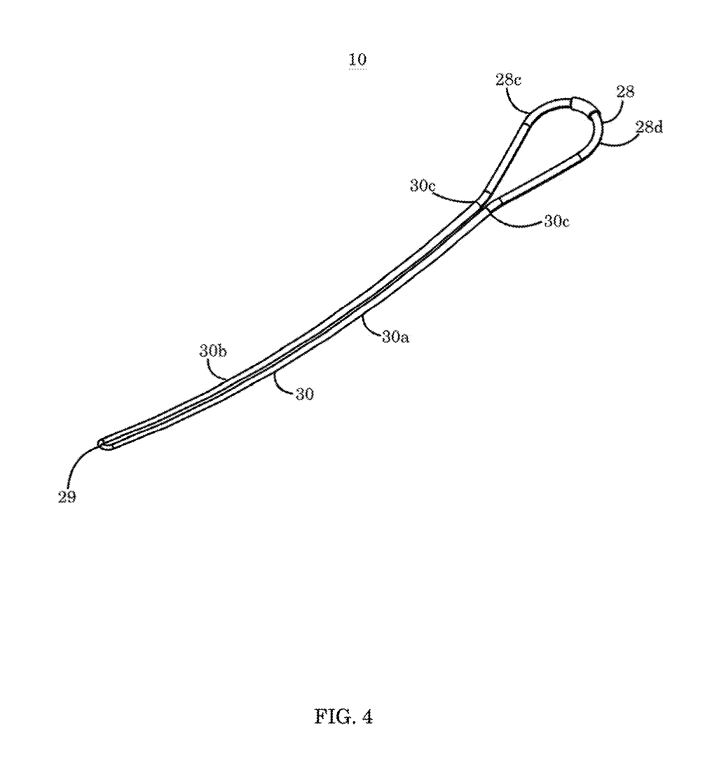

Reference is now made to FIGS. 1-9 in which a passer/retriever Device designated by reference numeral 10, in accordance with the invention is shown. Device 10 formed from a single continuous tubular shaped material which includes a dual shaft region 30, a U shape distal tip 29, and an outwardly expanded proximal region with permanently joined radius 28c and 28d end points 28a and 28b resulting in the proximal finger pull portion 28, with all afore mentioned elements in a permanently arced 30D2 form. The finger pull 28 provides a means to grip the Device for the purpose of advancing and retracting of the Device. The equally length dual shaft legs 30a and 30b of the tapered shaft 30, traversing proximally from the distal tip 29 and lying parallel to one another from the distal tip 29 then expand equally proximal of 30c in an outward direction 28e off the longitudinal centerline and terminating in an open radius 28c and 28d with end points 28a and 28b positioned suitable for joining. An un-bisected U shape distal tip 29 with the U centered on the minor diameter of a tapered shaft 30 the shaft having a first leg 30a and a second leg 30b. Wherein the interior radius 29a of the U shape distal tip 29 is sized relative to the maximum diameter of the taper in order to provide the correct U shape for the tip and maintain the two shaft legs 30a and 30b in a parallel configuration i.e. max taper diameter 0.10 the radius 29a=R0.05 min.

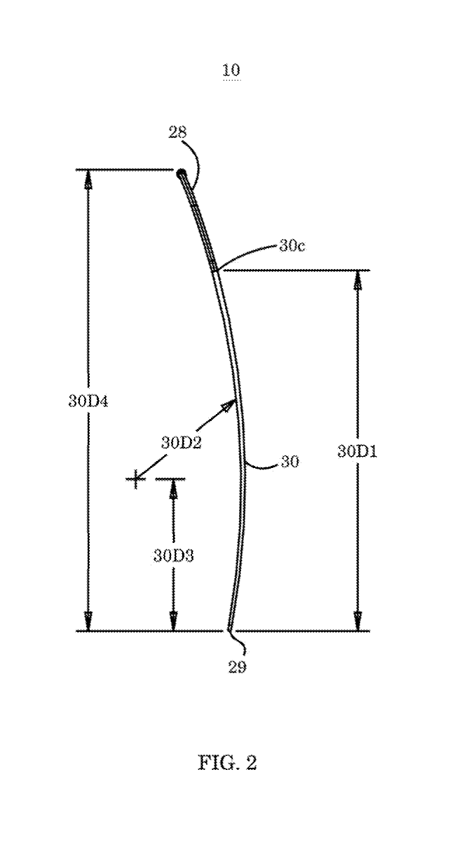

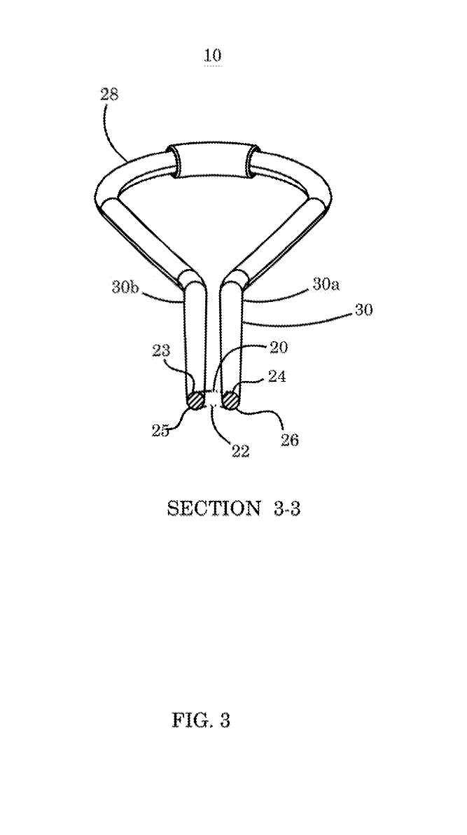

FIGS. 1-4 illustrates a tapered flexible dual shaft 30 the taper of the shaft legs 30a and 30b along length 30D1 is proportionate to the major diameter of the shaft to achieve the correct combination of stiffness and flexibility for the desired application. The taper centered on the longitudinal axis extends equally in both directions from the center of the U shape distal tip 29 to the start 30c of the proximal finger pull 28. FIG. 2 The dual shaft 30 and finger pull 28 region has a permanently formed arc 30D2 along length 30D4 the arc 30D2 not being laterally centered on the overall length of the Device is offset 30D3 from the distal tip of the Device and is sized to fit the applicable anatomy. FIG. 3 A transverse cross-section 3 having a naturally occurring curvature element which adapts to the upper 20 and under 22 surface. Curvature 20 is defined by a first surface 23 and a second surface 24 that is adjacent to the first surface 23. Curvature 22 is defined by a first surface 25 and a second surface 26 that is adjacent to the first surface 25.

FIG. 1 illustrates a U shaped aperture 31 in the distal tip 29 large enough to easily accommodate any applicable suture size. The benefit is having the aperture 31 at the very tip where it can be easily accessible, visually and physically, yet does not interfere with the function of the tip which works as a self-locating/guiding feature. Additionally the naturally rounded element of the tubular shape of the distal tip 29 and dual shaft 30 aids in limiting possible trauma during use.

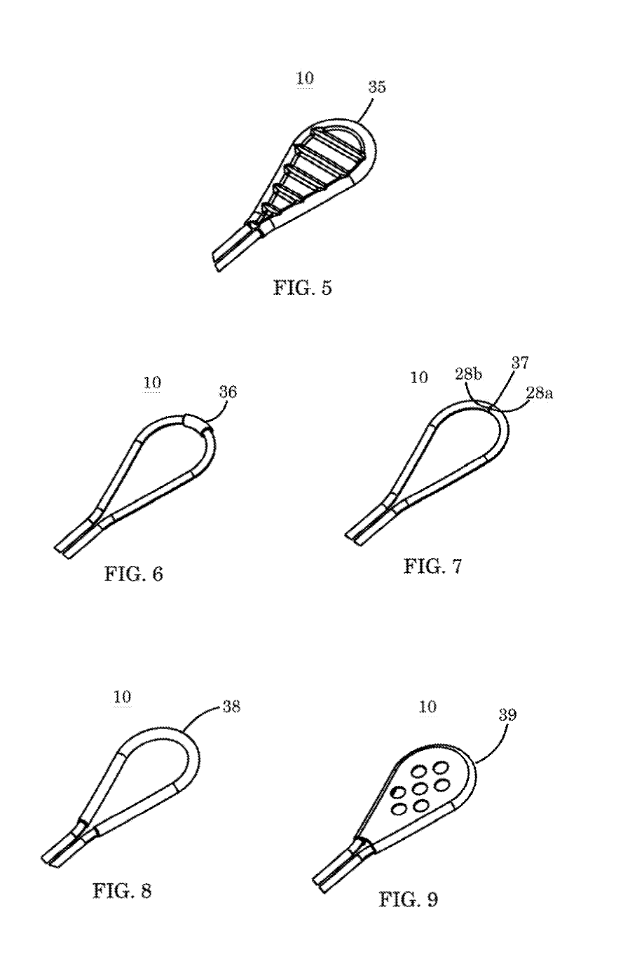

FIGS. 5 and 9 illustrates an additional embodiment of the invention with a solid finger pull suitable for thumb and index finger gripping 35 e.g. over-molded with a suitable material or 39 formed perforated metal. Additionally the over-mold and formed metal join the radius 28c and 28d end points 28a and 28b in a permanent fashion.

FIGS. 6 and 8 illustrates an additional embodiment of the invention with a short tubular component 36 providing the necessary amount of tubing to permanently join the finger pull radius 28c and 28d end points 28a and 28b: a longer tubular component or over-mold 38 providing coverage of the finger pull 28 to permanently join the finger pull radius 28c and 28d end points 28a and 28b.

FIG. 7 illustrates an additional embodiment of the invention with a weld 37 to permanently join the finger pull radius 28c and 28d end points 28a and 28b.

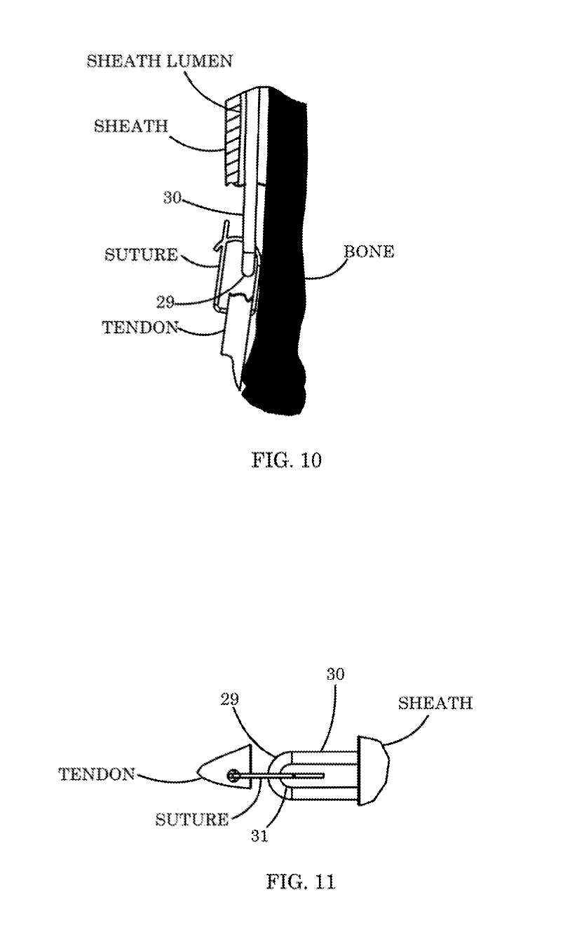

FIGS. 10 and 11 illustrate the Device 10 in a retrieval position resting within the tendon sheath lumen adjacent to the retracted free end of the severed tendon. Illustrating the retrieval of a suture which has been attached to the free end of a severed tendon the suture then being passed through the U shape distal tip 29 further illustrating the comparison of functionality and proportion between the suture and the U shaped aperture 31.

The foregoing description is considered as illustrative only of the principles of the invention. Further, since numerous modifications and changes may readily occur to those skilled in the art, it is not desired to limit the invention to the exact construction and operation shown and described, and accordingly, all suitable modifications and equivalents are considered to fall within the scope of the invention. Various features and advantages of the invention are set forth in the following claims.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

1. A passer/retriever Device, comprising:
a continuous single shaft which is permanently joined at the radii end points having two ends, a distal tip end and a proximal finger pull end;
a first leg and second leg shaft connected in a U shape at the distal tip end and longitudinally parallel then outward expanding proximally and connected at permanently joined radii end points of the proximal end;
said first leg of the shaft extending from the proximal radius end, tapering through a central region to be substantially parallel with the second leg of the shaft mirrorly extending from the proximal radius end, tapering through the central region where the shafts are proximally closest within the Device and connect at the distal end;
said shaft legs longitudinally arc from the extreme distal end through central region and through the extreme proximal end.

2. The Device of claim 1, wherein the proximal region has a filled inner portion making the aperture a solid and the radius ends permanently joined.

3. The Device of claim 1, wherein the proximal radius ends are joined with a non-metallic tubular connection portion making the ends permanently joined.

4. The Device of claim 1, wherein the proximal radius ends are welded making the ends permanently joined.

5. The Device of claim 1, wherein the distal region is a U shape with a minimum inner radius value not less than the maximum diameter of the shaft.

6. The Device of claim 1, wherein the Device consist of a flexible shape memory metal.

7. The Device of claim 3 wherein the metal is nickel titanium.

8. The Device of claim 1 wherein the shafts are thinner at the distal end and thicker at the proximal end.

9. The Device of claim 1 wherein the shafts are permanently joined at the proximal radius ends by means of a thin walled metal tube.

10. The Device of claim 1 wherein the shafts are permanently joined at the proximal radius ends by means of a weld.

11. The Device of claim 1 wherein the shafts are permanently joined at the proximal radius ends by means of a metal overlay crimped in place.

12. The Device of claim 1 wherein the shafts are permanently joined at the proximal radius ends by means of injection molded, over-mold of a steam sterilization stable material.

13. The Device of claim 1 wherein the shafts are permanently arced lying proximal of the distal tip.

14. The Device of claim 1, wherein the Device consists of a hollow tubular shaped flexible shape memory metal.

15. The Device of claim 1, wherein the Device consist of solid wire of a flexible shape memory metal.

16. The Device of claim 1, wherein the Device consist of a stainless steel metal.

17. A method for retrieval of a severed flexor tendon in the hand comprising: a Device having a flexible tapered dual shaft, a distal U shape end with aperture, a joined proximal finger pull end and a permanently formed longitudinal arc extending from the extreme distal end to the extreme proximal end. Once the severed flexor tendon repair site has been prepped for the repair procedure, the Device is introduced and advanced into the sheath lumen by gripping the proximal finger pull end. Then beginning at the repair site the shaft portion lying between the finger pull portion and distal tip is introduced, tip first into the tendon sheath lumen through the repair site opening and advanced through the flexor tendon sheath lumen to the retracted free end of the severed tendon. With the distal tip of the Device positioned adjacent to the severed tendon end, the surgeon attaches a suture to the severed tendon end and then the suture is passed through the aperture in the distal tip of the Device and secured to the Device. By means of the finger pull the Device, suture, and then tendon end are pulled/feed through the sheath lumen back to the repair site where the Device is then released from the suture and removed, the tendon repair procedure is then completed.
